(19) United States
(12) Patent Application Publication
(10) Pub. No.: US 2016/0279429 A1
(43) Pub. Date: Sep. 29, 2016

(54) METHOD AND APPARATUS FOR CONTROLLING TEMPORAL PATTERNS OF NEUROSTIMULATION

(71) Applicant: Boston Scientific Neuromodulation Corporation, Valencia, CA (US)

(72) Inventors: Bradley Lawrence Hershey, Valencia, CA (US); Michael A. Moffitt, Valencia, CA (US)

(21) Appl. No.: 15/079,340

(22) Filed: Mar. 24, 2016

Related U.S. Application Data
(60) Provisional application No. 62/137,567, filed on Mar. 24, 2015.

(51) Int. Cl. A61N 1/372; A61N 1/05

(52) U.S. Cl. CPC A61N 1/37247; A61N 1/0551

(57) ABSTRACT

An example of a neurostimulation system may include a storage device, a programming control circuit, and a graphical user interface (GUI). The storage device may store a set of one or more stimulation waveforms each associated with a stimulation field specified by a set of electrodes. Each stimulation waveform represents a pattern of neurostimulation pulses. The programming control circuit may be configured to generate stimulation parameters controlling delivery of the neurostimulation pulses according to the set of one or more stimulation waveforms. The GUI may include a composition control circuit configured to define each stimulation waveform as a function of one or more parameter graphs each depicted as a function of time. The composition control circuit may be configured to allow activation of one or more stimulation fields, present a parameter graph for each activated stimulation field, and allow for adjustment of each presented parameter graph.

### CLAIM OF PRIORITY

This application claims the benefit of priority under 35 U.S.C. §119(e) of U.S. Provisional Patent Application Ser. No. 62/137,567, filed on Mar. 24, 2015, which is herein incorporated by reference in its entirety.

### CROSS REFERENCE TO RELATED APPLICATIONS

This application is related to commonly assigned U.S. Provisional Patent Application Ser. No. 62/075,079, entitled “METHOD AND APPARATUS FOR PROGRAMMING COMPLEX NEUROSTIMULATION PATTERNS,” filed on Nov. 4, 2014, which is incorporated by reference in its entirety.

### TECHNICAL FIELD

This document relates generally to neurostimulation and more particularly to a method and system for programming a pattern of neurostimulation pulses using a graphical user interface (GUI).

### BACKGROUND

Neurostimulation, also referred to as neuromodulation, has been proposed as a therapy for a number of conditions. Examples of neurostimulation include Spinal Cord Stimulation (SCS), Deep Brain Stimulation (DBS), Peripheral Nerve Stimulation (PNS), and Functional Electrical Stimulation (FES). Implantable neurostimulation systems have been applied to deliver such a therapy. An implantable neurostimulation system may include an implantable neurostimulator, also referred to as an implantable pulse generator (IPG), and one or more implantable leads each including one or more electrodes. The implantable neurostimulator delivers neurostimulation energy through one or more electrodes placed on or near a target site in the nervous system. An external programming device is used to program the implantable neurostimulator with stimulation parameters controlling the delivery of the neurostimulation energy.

In one example, the neurostimulation energy is delivered in the form of electrical neurostimulation pulses. The delivery is controlled using stimulation parameters that specify spatial (where to stimulate), temporal (when to stimulate), and informational (patterns of pulses directing the nervous system to respond as desired) aspects of a pattern of neurostimulation pulses. Many current neurostimulation systems are programmed to deliver periodic pulses with one or a few uniform waveforms continuously or in bursts. However, the human nervous systems use neural signals having much more sophisticated patterns to communicate various types of information, including sensations of pain, pressure, temperature, etc. The nervous system may interpret an artificial stimulation with a simple pattern of stimuli as an unnatural phenomenon, and respond with an unintended and undesirable sensation and/or movement. For example, some neurostimulation therapies are known to cause paresthesia and/or vibration of non-targeted tissue or organ.

Recent research has shown that the efficacy and efficiency of certain neurostimulation therapies can be improved, and their side-effects can be reduced, by using patterns of neurostimulation pulses that emulate natural patterns of neural signals observed in the human body. While modern electronics can accommodate the need for generating such sophisticated pulse patterns, the capability of a neurostimulation system depends on its post-manufacturing programmability to a great extent. For example, a sophisticated pulse pattern may only benefit a patient when it is customized for that patient, and waveform patterns predetermined at the time of manufacturing may substantially limit the potential for the customization. In various applications, the customization may require pulse-by-pulse programmability of waveform parameters as well as electrode assignment, thereby making the programming of the pulse pattern a challenging task.

### SUMMARY

An example (e.g., “Example 1”) of a system for delivering neurostimulation using a plurality of electrodes may include a storage device, a programming control circuit, and a graphical user interface (GUI). The storage device may store a set of one or more stimulation waveforms each associated with a field of one or more stimulation fields. The one or more stimulation waveforms each represent a pattern of neurostimulation pulses to be delivered during a stimulation period. The one or more stimulation fields are each specified by a set of electrodes selected from the plurality of electrodes. The programming control circuit may be configured to generate a plurality of stimulation parameters controlling delivery of the neurostimulation pulses according to the set of one or more stimulation waveforms. The GUI may include a composition control circuit configured to define each stimulation waveform of the set of one or more stimulation waveforms as a function of one or more parameter graphs each depicted as a function of time for the stimulation period. The composition control circuit may include a waveform definition module configured to allow activation of one or more fields of the one or more stimulation fields, present a parameter graph of the one or more parameter graphs for each field of the activated one or more fields, and allow for adjustment of the presented each parameter graph.

In Example 2, the subject matter of Example 1 may optionally configured such that the composition control circuit further include a field definition module configured to define each field of the one or more stimulation fields by the set of electrodes selected from the plurality of electrodes and a current distribution over the selected set of electrodes.

In Example 3, the subject matter of any one or any combination of Examples 1 and 2 may optionally be configured such that the waveform definition module is configured to depict the presented each parameter graph as a curve.

In Example 4, the subject matter of any one or any combination of Examples 1-3 may optionally be configured such that the waveform definition module is configured to depict the presented each parameter graph as a pattern of symbols each representing one or more pulses of the neurostimulation pulses.

In Example 5, the subject matter of any one or any combination of Examples 1-4 may optionally be configured such that the waveform definition module is configured to present the one or more stimulation fields and receive a selection of the one or more fields for activation.

In Example 6, the subject matter of any one or any combination of Examples 1-5 may optionally be configured such that wherein the waveform definition module is configured allow the presented each parameter graph to be graphically edited.

In Example 7, the subject matter of any one or any combination of Examples 1-6 may optionally be configured such that the waveform definition module is configured to present one or more of a frequency graph, a duration graph, and an amplitude graph of the one or more parameter graphs. The frequency graph represents a pulse frequency, the duration graph representing a pulse width. The amplitude graph represents a pulse amplitude.

In Example 8, the subject matter of any one or any combination of Examples 1-7 may optionally be configured such that the storage device is configured to store a pattern library including one or more template waveforms, and the waveform definition module is further configured to select a template waveform from the template library as a stimulation waveform of the set of one or more stimulation waveforms or a parameter graph of the one or more parameter graphs.

In Example 9, the subject matter of any one or any combination of Examples 1-8 may optionally be configured such that the composition control circuit is configured to allow for export of a selected portion of the set of one or more stimulation waveforms from the system and import of a waveform into the system to be inserted to the set of the one or more stimulation waveforms.

In Example 10, the subject matter of any one or any combination of Examples 1-9 may optionally be configured to further include a stimulation device and a programming device. The stimulation device may include a stimulation output circuit configured to deliver the neurostimulation pulses and a stimulation control circuit configured to control the delivery of the neurostimulation pulses using the plurality of stimulation parameters. The programming device may be communicatively coupled to the stimulation device and include the storage device, the programming circuit, and the GUI.

In Example 11, the subject matter of Example 10 may optionally be configured such that the stimulation device includes an implantable stimulator including an implant telemetry circuit configured to receive the plurality of stimulation parameters, and the programming device includes an external programmer including an external telemetry circuit configured to transmit the plurality of stimulation parameters to the implantable stimulator via a wireless telemetry link.

An example of a method (e.g., “Example 12”) for controlling neurostimulation by a user for delivery through a plurality of electrodes is also provided. The method may include defining each stimulation waveform of a set of one or more stimulation waveforms as a function of one or more parameter graphs each depicted as a function of time for a stimulation period. The one or more stimulation waveforms are each associated with a field of one or more stimulation fields and representing a pattern of neurostimulation pulses to be delivered to the field during the stimulation period. The one or more stimulation fields are each specified by a set of electrodes selected from the plurality of electrodes. The method may further include allowing the user to activate one or more fields of the one or more stimulation fields using a graphical user interface (GUI), presenting a parameter graph of the one or more parameter graphs for each field of the activated one or more fields using the GUI, allowing the user to adjust the presented each parameter graph, and updating the set of one or more stimulation waveforms in response to the adjustment of the presented each parameter graph.

In Example 13, the subject matter of presenting the each parameter graph as found in Example 12 may optionally include presenting one or more of a frequency graph representing a pulse frequency, a duration graph representing a pulse width, and an amplitude graph representing a pulse amplitude.

In Example 14, the subject matter of presenting the each parameter graph as found in any one or any combination of Examples 12 and 13 may optionally include presenting one or more curves.

In Example 15, the subject matter of presenting the each parameter graph as found in any one or any combination of Examples 12-14 may optionally include presenting a pattern of symbols each representing one or more pulses of the neurostimulation pulses.

In Example 16, the subject matter of presenting the each parameter graph as found in any one or any combination of Examples 12-15 may optionally further include generating a plurality of stimulation parameters based on the set of the one or more stimulation waveforms. The plurality of stimulation parameters allow for delivery of the neurostimulation from a stimulation device to be controlled according to the set of the one or more stimulation waveforms.

In Example 17, the subject matter of defining the each stimulation waveform of the set of one or more stimulation waveforms as found in any one or any combination of Examples 12-16 may optionally include creating one or more template waveforms for the each stimulation waveform and selecting a template waveform from the stored one or more waveforms to be used as the each stimulation waveform.

In Example 18, the subject matter of creating the one or more template waveforms as found in Example 17 may optionally include creating one or more therapy-specific template waveforms for one or more types of neurostimulation therapy.

In Example 19, the subject matter of any one or any combination of Examples 12-18 may optionally include transmitting the plurality of stimulation parameters to an implantable stimulator and controlling delivery of the neurostimulation pulses from the implantable stimulator using the plurality of stimulation parameters.

This Summary is an overview of some of the teachings of the present application and not intended to be an exclusive or exhaustive treatment of the present subject matter. Further details about the present subject matter are found in the detailed description and appended claims. Other aspects of the disclosure will be apparent to persons skilled in the art upon reading and understanding the following detailed description and viewing the drawings that form a part thereof, each of which are not to be taken in a limiting sense. The scope of the present disclosure is defined by the appended claims and their legal equivalents.

### BRIEF DESCRIPTION OF THE DRAWINGS

The drawings illustrate generally, by way of example, various embodiments discussed in the present document. The drawings are for illustrative purposes only and may not be to scale.

FIG. 1 illustrates an embodiment of a neurostimulation system.

FIG. 2 illustrates an embodiment of a stimulation device and a lead system, such as may be implemented in the neurostimulation system of FIG. 1.

FIG. 3 illustrates an embodiment of a programming device, such as may be implemented in the neurostimulation system of FIG. 1.

FIG. 4 illustrates an embodiment of an implantable neurostimulation system and portions of an environment in which the implantable neurostimulation system may be used.

FIG. 5 illustrates an embodiment of an implantable stimulator and one or more leads of an implantable neurostimulation system, such as the implantable neurostimulation system of FIG. 4.

FIG. 6 illustrates an embodiment of an external programmer of an implantable neurostimulation system, such as the implantable neurostimulation system of FIG. 4

FIG. 7 illustrates an embodiment of portions of a circuit of a graphical user interface (GUI) of the external programmer.

FIG. 8 illustrates an embodiment of portions of a screen of the GUI for defining one or more stimulation waveforms.

FIG. 9 illustrates an embodiment of a method for specifying a segment of a stimulation waveform for repetition.

FIG. 10 illustrates an embodiment of a method for defining a stimulation waveform.

FIG. 11 illustrates an embodiment of portions of the circuit of the GUI providing for various viewing/editing controls using the GUI.

FIG. 12 illustrates an embodiment of portions of the screen of the GUI allowing for access to the various viewing/editing controls.

### DETAILED DESCRIPTION

In the following detailed description, reference is made to the accompanying drawings which form a part hereof, and in which is shown by way of illustration specific embodiments in which the invention may be practiced. These embodiments are described in sufficient detail to enable those skilled in the art to practice the invention, and it is to be understood that the embodiments may be combined, or that other embodiments may be utilized and that structural, logical and electrical changes may be made without departing from the spirit and scope of the present invention. References to “an”, “one”, or “various” embodiments in this disclosure are not necessarily to the same embodiment, and such references contemplate more than one embodiment. The following detailed description provides examples, and the scope of the present invention is defined by the appended claims and their legal equivalents.

This document discusses a method and system for programming neurostimulation pulse patterns using a graphical user interface (GUI). Advancements in neuroscience and neurostimulation research have led to a demand for using complex and/or individually optimized patterns of neurostimulation pulses for various types of therapies. The capability of a neurostimulation system in treating various types of disorders will be limited by the programmability of such patterns of neurostimulation pulses. Because such a task may be performed at least in part by a user such as a physician or other caregiver with the patient in a clinical setting, there is a need for an intuitive, user-friendly method and system for programming the neurostimulation pulse pattern.

The present system allows for definition of a pattern of neurostimulation pulses using a graphical method. In various embodiments, the GUI allows the pattern of neurostimulation pulses to be defined as a set one or more stimulation waveforms each to be delivered to a stimulation field specified by a set of electrodes. The one or more stimulation waveforms are defined using various parameters each specified as a function of time and presented as an editable graph. In various embodiments, defining a complex pattern of neurostimulation pulses using a few graphs representing variable parameters substantially reduces the amount of user interaction required to create and adjust the pattern, for example when compared to an existing method of tedious control of various parameters for numerous pulses in the pattern. The reduced amount of user interaction may allow changes to the pattern of neurostimulation pulses to be made in real time.

In various embodiments, the present system facilitates creation and adjustment of complex patterns of neurostimulation pulses, such as a pattern that includes sequences of pulses that have different amplitude and timing parameters and are to be delivered using different combinations of electrodes. The GUI allows for programming of the amplitude and timing parameters for each of one or more stimulation fields. The amplitude and timing parameters are each depicted as a parameter graph being a function of time. Examples of the amplitude and timing parameters that can be depicted as parameter graphs include pulse frequency, pulse amplitude, and pulse width. The one or more stimulation fields are each specified by a set of electrodes through which a neurostimulation pulse is delivered. In various embodiments, the one or more stimulation fields are programmable for current steering (i.e. electrode fractionalization).

In various embodiments, the present system enable fast and intuitive definition of patterns of neurostimulation pulses in a way that allows the patterns to be designed, tested, and modified in real time. While stimulation waveforms generated from frequency, duration (pulse width), and/or amplitude graphs are discussed as specific examples, the present subject matter allows for definition of a pattern of neurostimulation pulses using one or more stimulation waveforms generated by any one or more graphs each representing a parameter that can be specified as a function of time. While the application in neurostimulation is discussed as a specific example, the present subject matter allows for waveform definition in any electrical stimulation and other medical device applications in which a waveform representing a pattern or sequence of pulses is to be defined.

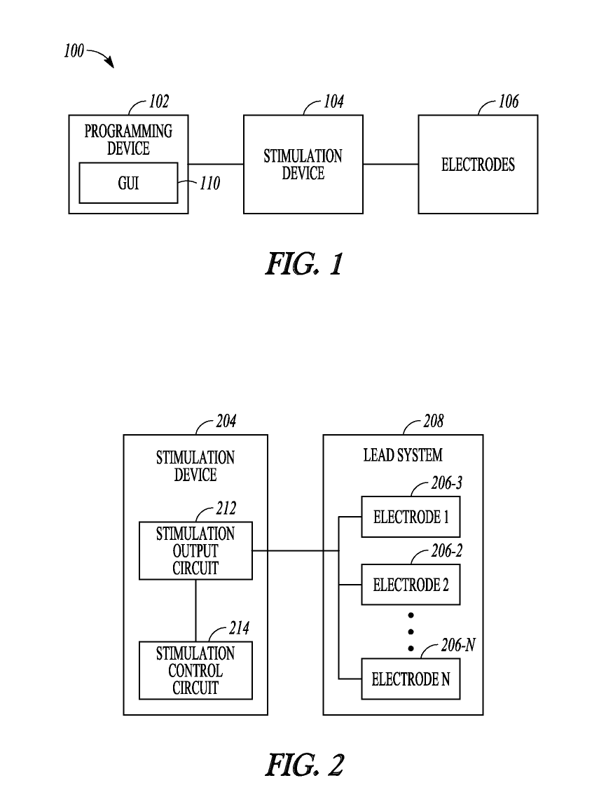

FIG. 1 illustrates an embodiment of a neurostimulation system 100.

System 100 includes electrodes 106, a stimulation device 104, and a programming device 102. Electrodes 106 are configured to be placed on or near one or more neural targets in a patient. Stimulation device 104 is configured to be electrically connected to electrodes 106 and deliver neurostimulation energy, such as in the form of electrical pulses, to the one or more neural targets though electrodes 106. The delivery of the neurostimulation is controlled by using a plurality of stimulation parameters, such as stimulation parameters specifying a pattern of the electrical pulses and a selection of electrodes through which each of the electrical pulses is delivered. In various embodiments, at least some parameters of the plurality of stimulation parameters are programmable by a user, such as a physician or other caregiver who treats the patient using system 100. Programming device 102 provides the user with accessibility to the user-programmable parameters. In various embodiments, programming device 102 is configured to be communicatively coupled to stimulation device via a wired or wireless link.

In various embodiments, programming device 102 includes a graphical user interface (GUI) 110 that allows the user to set and/or adjust values of the user-programmable parameters by creating and/or editing graphical representations of various waveforms. Such waveforms may include, for example, a waveform representing a pattern of neurostimulation pulses to be delivered to the patient as well as individual waveforms that are used as building blocks of the pattern of neurostimulation pulses, such as the waveform of each pulse in the pattern of neurostimulation pulses. The GUI may also allow the user to set and/or adjust stimulation fields each defined by a set of electrodes through which one or more neurostimulation pulses represented by a waveform are delivered to the patient. The stimulation fields may each be further defined by the distribution of the current of each neurostimulation pulse in the waveform. In various embodiments, neurostimulation pulses for a stimulation period (such as the duration of a therapy session) may be delivered to multiple stimulation fields, and the overall pattern of neurostimulation pulses may be represented by a set of stimulation waveforms each associated (i.e., to be delivered to) a field of the multiple stimulation fields.

In various embodiments, GUI 110 allows the user to define a set of one or more stimulation waveforms each associated with a field of one or more stimulation fields. The one or more stimulation waveforms each represent a pattern of neurostimulation pulses to be delivered during a stimulation period. The one or more stimulation fields are each specified by at least one or more electrodes of electrodes 106. The set of one or more stimulation waveforms represent the pattern of neurostimulation pulses to be delivered to the patient from stimulation device 104 through the one or more electrodes selected from electrodes 106 during the stimulation period.

FIG. 2 illustrates an embodiment of a stimulation device 204 and a lead system 208, such as may be implemented in neurostimulation system 100. Stimulation device 204 represents an embodiment of stimulation device 104 and includes a stimulation output circuit 212 and a stimulation control circuit 214. Stimulation output circuit 212 produces and delivers neurostimulation pulses. Stimulation control circuit 214 controls the delivery of the neurostimulation pulses using the plurality of stimulation parameters, which specifies a pattern of the neurostimulation pulses. Lead system 208 includes one or more leads each configured to be electrically connected to stimulation device 204 and a plurality of electrodes 206 distributed in the one or more leads. The plurality of electrodes 206 includes electrode 206-1, electrode 206-2, . . . electrode 206-N, each a single electrically conductive contact providing for an electrical interface between stimulation output circuit 212 and tissue of the patient, where N≧2. The neurostimulation pulses are each delivered from stimulation output circuit 212 through a set of electrodes selected from electrodes 206. In various embodiments, the neurostimulation pulses may include one or more individually defined pulses, and the set of electrodes may be individually definable by the user for each of the individually defined pulses or each of collections of pulse intended to be delivered using the same combination of electrodes.

In various embodiments, the number of leads and the number of electrodes on each lead depend on, for example, the distribution of target(s) of the neurostimulation and the need for controlling the distribution of electric field at each target. In one embodiment, lead system 208 includes 2 leads each having 8 electrodes.

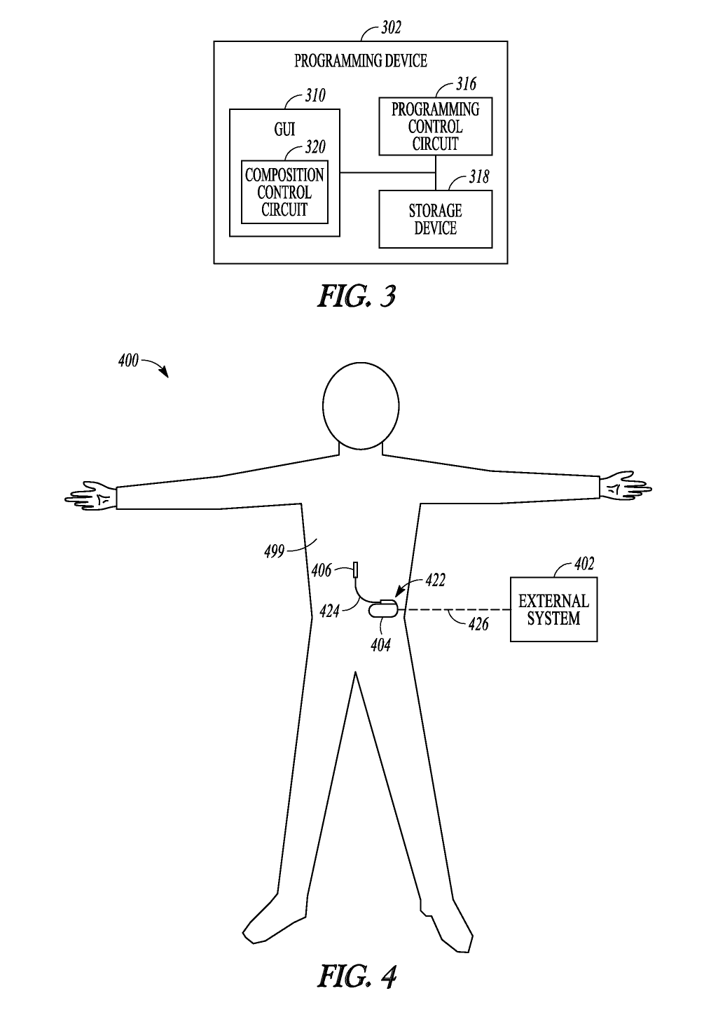

FIG. 3 illustrates an embodiment of a programming device 302, such as may be implemented in neurostimulation system 100. Programming device 302 represents an embodiment of programming device 102 and includes a storage device 318, a programming control circuit 316, and a GUI 310. Storage device 318 stores a set of one or more stimulation waveforms each associated with a field of one or more stimulation fields. The one or more stimulation waveforms each represent a pattern of neurostimulation pulses to be delivered during a stimulation period. The one or more stimulation fields are each specified by a set of electrodes through which the associated stimulation waveform is to be delivered. Programming control circuit 316 generates the plurality of stimulation parameters that controls the delivery of the neurostimulation pulses according to the set of one or more stimulation waveforms. GUI 310 represents an embodiment of GUI 110 and includes a composition control circuit 320 that allows for definition of the pattern of neurostimulation pulses by creating and/or adjusting each waveform of the set of one or more stimulation waveforms using a graphical method. In various embodiments, GUI 310 allows the user to define each waveform of the set of one or more stimulation waveforms as a function of one or more parameter graphs each being a function of time during the stimulation period. The one or more parameter graphs each represent a user-programmable parameter of the plurality of stimulation parameters. Composition control circuit 320 is configured to present the one or more stimulation fields, allow for activation of each field of the one or more stimulation fields, present one or more parameter graphs for each activated field, allow for adjustment of each graph of the presented one or more parameter graphs, and update the set of one or more stimulation waveforms in response to the adjustment of any of the one or more parameter graphs.

In various embodiments, GUI 310 includes any type of presentation device, such as interactive or non-interactive screens, and any type of user input devices that allow the user to edit the graphical representation of the accessed waveform, such as touchscreen, keyboard, keypad, touchpad, trackball, joystick, and mouse. In one embodiment, GUI 310 includes an interactive touchscreen and displays the graphical representation of the accessed waveform by visually indicating parameters defining the accessed waveform on the touchscreen. The touchscreen allows the user to modify one or more parameters of the visually indicated parameters, such as by dragging one or more segments of the accessed waveform.

In various embodiments, circuits of neurostimulation 100, including its various embodiments discussed in this document, may be implemented using a combination of hardware and software. For example, the circuit of GUI 110, stimulation control circuit 214, programming control circuit 316, and composition control circuit 320, including their various embodiments discussed in this document, may be implemented using an application-specific circuit constructed to perform one or more particular functions or a general-purpose circuit programmed to perform such function(s). Such a general-purpose circuit includes, but is not limited to, a microprocessor or a portion thereof, a microcontroller or portions thereof, and a programmable logic circuit or a portion thereof.

FIG. 4 illustrates an implantable neurostimulation system 400 and portions of an environment in which system 400 may be used. System 400 includes an implantable system 422, an external system 402, and a telemetry link 426 providing for wireless communication between implantable system 422 and external system 402. Implantable system 422 is illustrated in FIG. 4 as being implanted in a patient's body 499.

Implantable system 422 includes an implantable stimulator (also referred to as an implantable pulse generator, or IPG) 404, a lead system 424, and electrodes 406, which represent an embodiment of stimulation device 204, lead system 208, and electrodes 206, respectively. External system 402 represents an embodiment of programming device 302. In various embodiments, external system 402 includes one or more external (non-implantable) devices each allowing the user and/or the patient to communicate with implantable system 422. In some embodiments, external 402 includes a programming device intended for the user to initialize and adjust settings for implantable stimulator 404 and a remote control device intended for use by the patient. For example, the remote control device may allow the patient to turn implantable stimulator 404 on and off and/or adjust certain patient-programmable parameters of the plurality of stimulation parameters.

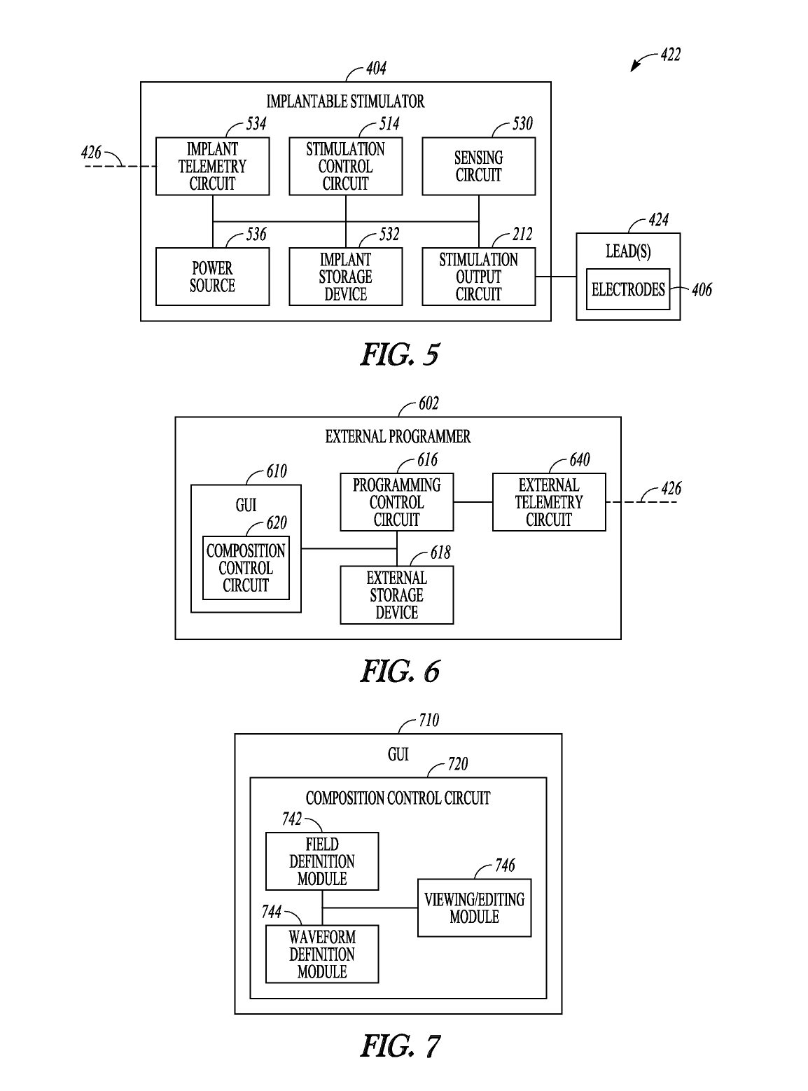

FIG. 5 illustrates an embodiment of implantable stimulator 404 and one or more leads 424 of an implantable neurostimulation system, such as implantable system 422. Implantable stimulator 404 may include a sensing circuit 530 that is optional and required only when the stimulator has a sensing capability, stimulation output circuit 212, a stimulation control circuit 514, an implant storage device 532, an implant telemetry circuit 534, and a power source 536. Sensing circuit 530, when included and needed, senses one or more physiological signals for purposes of patient monitoring and/or feedback control of the neurostimulation. Examples of the one or more physiological signals include neural and other signals each indicative of a condition of the patient that is treated by the neurostimulation and/or a response of the patient to the delivery of the neurostimulation. Stimulation output circuit 212 is electrically connected to electrodes 406 through lead 424, and delivers each of the neurostimulation pulses through a set of electrodes selected from electrodes 406. Stimulation control circuit 514 represents an embodiment of stimulation control circuit 214 and controls the delivery of the neurostimulation pulses using the plurality of stimulation parameters specifying the pattern of neurostimulation pulses. In one embodiment, stimulation control circuit 514 controls the delivery of the neurostimulation pulses using the one or more sensed physiological signals. Implant telemetry circuit 534 provides implantable stimulator 404 with wireless communication with another device such as a device of external system 402, including receiving values of the plurality of stimulation parameters from external system 402. Implant storage device 532 stores values of the plurality of stimulation parameters. Power source 536 provides implantable stimulator 404 with energy for its operation. In one embodiment, power source 536 includes a battery. In one embodiment, power source 536 includes a rechargeable battery and a battery charging circuit for charging the rechargeable battery. Implant telemetry circuit 534 may also function as a power receiver that receives power transmitted from external system 402 through an inductive couple.

In various embodiments, sensing circuit 530 (if included), stimulation output circuit 212, stimulation control circuit 514, implant telemetry circuit 534, implant storage device 532, and power source 536 are encapsulated in a hermetically sealed implantable housing. In various embodiments, lead(s) 424 are implanted such that electrodes 406 are places on and/or around one or more targets to which the neurostimulation pulses are to be delivered, while implantable stimulator 404 is subcutaneously implanted and connected to lead(s) 424 at the time of implantation.

FIG. 6 illustrates an embodiment of an external programmer 602 of an implantable neurostimulation system, such as external system 402 of system 400. External programmer 602 represents an embodiment of programming device 302, and includes an external telemetry circuit 640, an external storage device 618, a programming control circuit 616, and a GUI 610.

External telemetry circuit 640 provides external programmer 602 with wireless communication with another device such as implantable stimulator 404 via telemetry link 426, including transmitting the plurality of stimulation parameters to implantable stimulator 404. In one embodiment, external telemetry circuit 640 also transmits power to implantable stimulator 404 through an inductive couple.

External storage device 618 stores one or more waveform sets each including a set of one or more stimulation waveforms for delivery during a neurostimulation therapy session having a stimulation period. The one or more stimulation waveforms are each associated with a field of one or more stimulation fields and representing a pattern of neurostimulation pulses to be delivered to that field during the stimulation period. In various embodiments, each of the one or more waveform sets can be selected for modification by the user and/or for use in programming a stimulation device such as implantable stimulator 404 to deliver a therapy. In this document, the “set of one or more stimulation waveforms” may include a waveform set selected from external storage device 618 or a new waveform set to be added to the one or more waveform sets stored in external storage device 618.

In various embodiments, each waveform in the set of one or more stimulation waveforms is definable on a pulse-by-pulse basis, and external storage device 618 may include a pulse library that stores one or more individually definable pulse waveforms each defining a pulse type of one or more pulse types. External storage device 618 also stores one or more individually definable fields. Each waveform in the set of one or more stimulation waveforms is associated with a field of the one or more individually definable fields. Each field of the one or more individually definable fields is defined by a set of electrodes through which neurostimulation pulses of one stimulation waveform of the set of one or more stimulation waveforms is delivered. In various embodiments, each field of the one or more individually definable fields is defined by the set of electrodes through which the pulse of the neurostimulation pulses is delivered and a current distribution of the pulse over the set of electrodes.

Programming control circuit 616 represents an embodiment of programming control circuit 316 and generates the plurality of stimulation parameters, which is to be transmitted to implantable stimulator 404, based on the pattern of neurostimulation pulses as represented by the set of the one or more stimulation waveforms. The pattern may be created and/or adjusted by the user using GUI 610 and stored in external storage device 618. In various embodiments, programming control circuit 616 checks values of the plurality of stimulation parameters against safety rules to limit these values within constraints of the safety rules. In one embodiment, the safety rules are heuristic rules.

GUI 610 represents an embodiment of GUI 310 and allows the user to define the pattern of neurostimulation pulses by creating and/or adjusting the set of one or more stimulation waveforms representing the pattern. GUI 610 defines each waveform of the set of one or more stimulation waveforms as a function of one or more parameter graphs each being a function of time during the stimulation period. The one or more parameter graphs each represent a user-programmable parameter. Examples of the one or more parameter graphs include a frequency graph representing a pulse frequency, a duration graph representing a pulse width, and an amplitude graph representing a pulse amplitude. In various embodiments, GUI 610 allows for selection of a stimulation field and allows for creation and/or adjustment of the stimulation waveform associated with the selected stimulation field. In various embodiments, the one or more parameter graphs may each be depicted in one or more ways to facilitate the user's viewing and understanding of the pattern of neurostimulation patter. In one embodiment, the one or more parameter graphs are each depicted as a curve. In another embodiment, the one or more parameter graphs are each depicted as a pattern of pulses, with each pulse in the pattern of pulses representing one or more pulses of the neurostimulation pulses. In another embodiment, both the curve and the pattern of pulses are presented to depict the same parameter graph.

GUI 610 includes a composition control circuit 620, which represents an embodiment of composition control circuit 320. Composition control circuit 620 allows the user to activate one or more fields, presents each parameter graph for each activated field, allows the user to adjust the presented parameter graph, and updates the stimulation waveform associated with the activated field in response to the adjustment of the presented parameter graph. The set of one or more stimulation waveforms are updated after this is repeated for each of the one or more stimulation fields specified for the neurostimulation therapy session. In various embodiments, composition control circuit 620 also allows the user to create and/adjust the one or more stimulation fields.

In various embodiments, external programmer 602 has operation modes including a composition mode and a real-time programming mode. Under the composition mode (also known as the pulse pattern composition mode), composition control circuit 620 is activated, while programming control circuit 616 is inactivated. Programming control circuit 616 does not dynamically updates values of the plurality of stimulation parameters in response to any change in the set of one or more stimulation waveforms. Under the real-time programming mode, both composition control circuit 620 and programming control circuit 616 are activated. Programming control circuit 616 dynamically updates values of the plurality of stimulation parameters in response to changes in the set of one or more stimulation waveforms, and transmits the plurality of stimulation parameters with the updated values to implantable stimulator 404.

FIG. 7 illustrates an embodiment of portions of a circuit of a GUI 710, which represents an embodiment of GUI 610 and includes a composition control circuit 720. Composition control circuit 720 represents an embodiment of composition circuit 620 and includes a field definition module 742, a waveform definition module 744, and a viewing/editing module 746.

Field definition module 742 defines the one or more stimulation fields associated with the set of one or more stimulation waveforms for the neurostimulation therapy session having the stimulation period. In various embodiments, field definition module 742 allows the user to specify a set of electrodes used for delivering the neurostimulation pulses of each waveform of the set of one or more stimulation waveforms. In various embodiments, field definition module 742 allows the user to specify the set of electrodes used for delivering the neurostimulation pulses of each waveform of the set of one or more stimulation waveforms and the current distribution over the electrodes (such as by specifying a percentage of the current associated with each electrode). In various embodiments, various predefined fields are stored in external storage device 618, and field definition module 742 allows the user to specify the electrodes by selecting one or more fields from the predefined fields stored in external storage device 618. In various embodiments, field definition module 742 allows the user to select one or more fields from the predefined fields stored in external storage device 618 for use as one or more templates and create one or more new fields by adjusting the one or more templates.

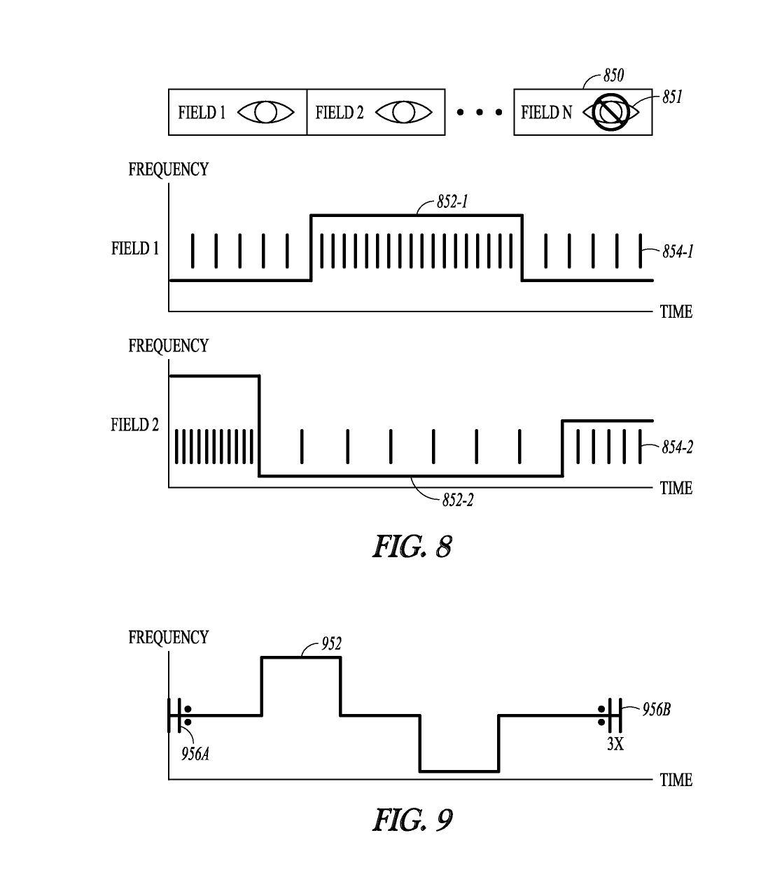

Waveform definition module 744 defines each stimulation waveform of the set of one or more stimulation waveforms. FIG. 8 illustrates an embodiment of portions of a screen of GUI 710 as controlled by waveform definition module 744 for defining stimulation waveforms. In various embodiments, waveform definition module 744 allows the user to select one or more fields from the one or more stimulation fields specified for the neurostimulation therapy session. In one embodiment, as illustrated in FIG. 8, waveform definition module 744 presents N stimulation fields 850 each including a visibility symbol (e.g., an “eye” as illustrated in FIG. 8) on the screen, and receives from the user a selection of one or more fields for activation (e.g., fields 1 and 2 selected as illustrated in FIG. 8). For example, the user may select by using a computer mouse or touchpad to move a cursor to visibility symbol 851 associated with the field to be selected and click a mouse or tap on the touch pad. In response to the activation of the one or more fields, waveform definition module 744 presents each parameter graph of the one or more parameter graphs for each field of the activated one or more fields. For example, as illustrated in FIG. 8, frequency graphs are displayed for the activated fields 1 and 2. In the illustrated example, the frequency graphs are depicted as a curve 852-1 for field 1 and a curve 852-2 for field 2, as well as a pulse pattern 854-1 for field 1 and a pulse pattern 854-2. In various embodiments, waveform definition module 744 may allow the user to choose to display curves 852, pulse patterns 854, or both curves 852 and pulse patterns 854, depending the user's preference. For example, some users may find the frequency graph easier to view and/or understand when being displayed as the pulse pattern as compared to the curve, and easier to measure and/or adjust when being displayed as the curve as compared to the pulse pattern. Other ways of representing the parameter graphs may be applied without departing from the scope of the present subject matter.

While the frequency graph is illustrated in FIG. 8, other parameter graphs may include a duration graph representing a pulse width and an amplitude graph representing a pulse amplitude. In various embodiments, waveform definition module 744 may present a parameter graph for any parameter that can be expressed as a function of time to allow the user to adjust that parameter by graphically editing the parameter graph. When multiple fields are activated (e.g., fields 1 and 2 are activated as illustrated in FIG. 8), the parameter graphs (pulse patterns, and other field-specific displayed features) for each field may be displayed in a space designated for that field (as illustrated in FIG. 8), displayed in a color designated for that field, or displayed using a line style designated for that field.

In one embodiment, each displayed parameter graph is adjustable by the user. In another embodiment, each displayed parameter graph is made selectable for adjustment. In other words, waveform definition module 744 may be configured to receive a user selection of a displayed parameter graph and unlock the selected parameter graph to allow for adjustment by the user. This reduces the possibility of accidental adjustments.

In various embodiments, waveform definition module 744 allows the user to apply graphical indicators on a parameter graph to denote specified stimulation patterns. For example, FIG. 9 illustrates an embodiment of specifying a segment of a stimulation waveform for repetition. In the illustrated embodiment, repeat signs (as used in musical composition) are placed on a frequency graph to specify a segment of the frequency graph to be repeated, and a repetition sign associated with the repeat sign is used to specify the number of repetitions. For example, “3×” means that the segment of frequency curve between the repeat signs is to be repeated three times.

Waveform definition module 744 updates the stimulation waveform defined by the parameter graph in response to adjustments made to the parameter graph. The set of one or more stimulation waveforms will be completely updated upon completion of all the parameter graph adjustments.

In one embodiment, waveform definition module 744 provides the user with a template waveform that can be adjusted to create a new stimulation waveform or new set of one or more stimulation waveforms. The template waveform is a predefined stimulation waveform or predefined set of one or more stimulation waveforms. As the stimulation waveform is defined as a function of one or more parameter graphs, the template waveform may include one or more templates for the one or more parameter graphs. External storage device 618 can include a pattern library including one or more template waveforms. In one embodiment, the pattern library may include one or more template waveforms created for one or more types of neurostimulation therapies and/or one or more types of disorders. Waveform definition module 744 allows the user to select a template waveform from the template library and present the selected template waveform as the one or more parameter graphs. In one embodiment, waveform definition module 744 allows the user to create template waveforms. For example, waveform definition module 744 may allow the user to adjust a template waveform and add the adjusted template waveform as a new template waveform to the one or more template waveforms of the pattern library.

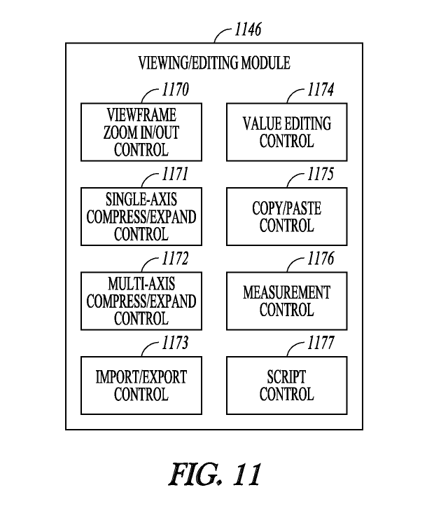

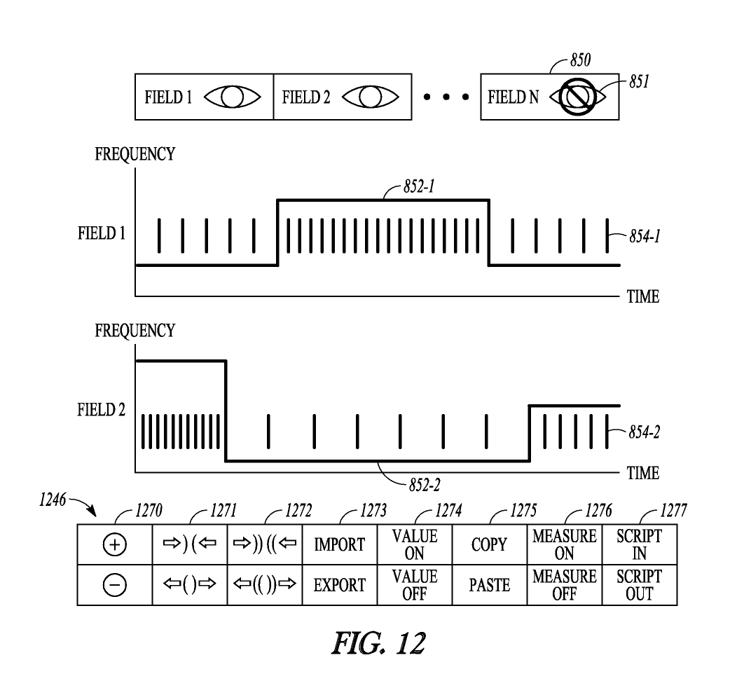

Viewing/editing module 746 allows the user to use various features of GUI 710 to facilitate, for example, viewing and graphically adjusting parameter graphs and sharing stimulation waveforms and/or parameter graphs with other users. Examples of such features are discussed below with reference to FIGS. 11 and 12.

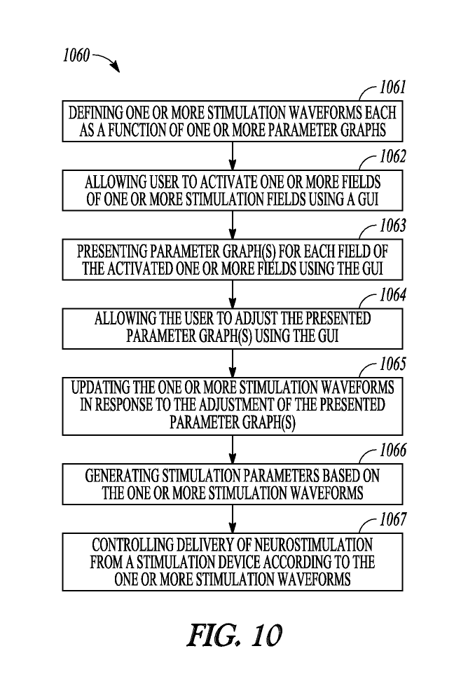

FIG. 10 illustrates an embodiment of a method 1060 for defining a set of one or more stimulation waveforms representing a pattern of neurostimulation pulses to be delivered during a neurostimulation therapy session having a stimulation period. In one embodiment, method 1060 is performed using system 100, including its various embodiments as discussed in this document. In one embodiment, GUI 110, 310, 610, or 710 is configured for performing method 1060. FIG. 8 as discussed above illustrates an embodiment of portion of a GUI, such as GUI 110, 310, 610, or 710 for displaying parameter graphs, among other things, when method 1060 is performed.

At 1061, the set of one or more stimulation waveforms is defined. The one or more stimulation waveforms are each defined as a function of one or more parameter graphs each depicted as a function of time for the stimulation period. The one or more parameter graphs each represent a user-programmable parameter. The one or more stimulation waveforms are each associated with a field of one or more stimulation fields and representing a pattern of neurostimulation pulses to be delivered to that field during the stimulation period. The one or more stimulation fields are each specified by a set electrodes selected from the plurality of electrodes. In one embodiment, the one or more stimulation fields are further specified by a current distribution over the selected set of electrodes.

In various embodiments, one or more template waveforms may be created to be the set of one or more stimulation waveforms or any stimulation waveform in the set. Defining the set of one or more stimulation waveforms or defining any one of the one or more stimulation waveforms may include selecting a template waveform from the stored one or more template waveforms. A template waveform is a predefined stimulation waveform representing a predefined pattern of neurostimulation pulses. External storage device 618 may include a pattern library including one or more basic template waveforms and/or one or more therapy-specific template waveforms. A basic template waveform serves as a beginning point, for example, for the user to create a new custom pattern of neurostimulation pulses by performing method 1060. A therapy-specific template waveform serves as a default waveform, for example, for a specific type of neurostimulation therapy that may be used to treat a patient with or without modification as determined by the user. When modification is deemed necessary or desirable, method 1060 may be performed.

At 1062, the user is allowed to activate one or more fields of the one or more stimulation fields using the GUI. Because the one or more stimulation waveforms are each associated with a field of the one or more stimulation fields, a field is to be activated by the use for viewing and/or adjusting the stimulation waveform associated with that field.

At 1063, each parameter graph of the one or more parameter graphs for the activated one or more fields are presented on the screen of the GUI. In one embodiment, the GUI allows the user to select each of the one or more parameter graphs for display and/or adjustment. In various embodiments, the one or more parameter graphs may include a frequency graph representing a pulse frequency, a duration graph representing a pulse width, an amplitude graph representing a pulse amplitude, and/or any other stimulation parameter that may be defined as a function of time for the stimulation period. In various embodiments, each parameter graph may be presented as a curve (such as curves 852-1 and 852-2 as illustrated in FIG. 8), a pattern of symbols each representing one or more pulses of the neurostimulation pulses (such as patterns 854-1 and 854-2 as illustrated in FIG. 8), a combination of the curve and the pattern, or any other representation of the underlying parameter. In one embodiment, the user is allowed to select one or more particular symbols for presentation based on personal preference for easy viewing and understanding.

At 1064, the user is allowed to adjust each of the presented one or more parameter graphs using the GUI. In one embodiment, each parameter graph when displayed is adjustable by the user. In another embodiment, the user is required to select each presented parameter graph for adjustment. In various embodiments, the presented one or more parameter graphs are adjustable by graphical editing. An example of adjusting parameter curves by graphical editing is discussed in U.S. Provisional Patent Application Ser. No. 62/075,079, entitled “METHOD AND APPARATUS FOR PROGRAMMING COMPLEX NEURO STIMULATION PATTERNS,” filed on Nov. 4, 2014, assigned to Boston Scientific Neuromodulation Corporation, which is incorporated by reference in its entirety.

At 1065, the set of one or more stimulation waveforms is updated in response to the adjustment of the presented one or more parameter graphs. In one embodiment, the set of one or more stimulation waveforms is updated automatically upon the each change made by the user. In another embodiment, the set of one or more stimulation waveforms is updated in response to an update command entered by the user.

At 1066, a plurality of stimulation parameters is generated based on the set of the one or more stimulation waveforms. In other words, the stimulation waveform is converted into a plurality of stimulation parameters. The plurality of stimulation parameters allows for delivery of the neurostimulation from a stimulation device to be controlled according to the set of the one or more stimulation waveforms. In one embodiment, such as under the composition mode of external programmer 602, the plurality of stimulation parameters is generated in response to a command entered by the user after completing a process of defining the set of one or more stimulation waveforms. In another embodiment, such as under the real-time programming mode of external programmer 602, the plurality of stimulation parameters is generated in response to each change (or each predetermined type of change) in the set of one or more stimulation waveforms, such as in response to each adjustment of a parameter graph during the process of defining the set of one or more stimulation waveforms.

At 1067, delivery of neurostimulation from a stimulation device is controlled according to the set of one or more stimulation waveforms. The plurality of stimulation parameters is transmitted to the stimulation device, such as stimulation device 104 or 204, and the delivery of the neurostimulation pulses from the stimulation device is controlled using the plurality of stimulation parameters. In one embodiment, the plurality of stimulation parameters is transmitted to an implantable stimulator from an external programmer via telemetry, such as being transmitted to implantable stimulator 404 from external programmer 602 via telemetry link 426, and the delivery of the neurostimulation pulses from the implantable stimulator is controlled using the plurality of stimulation parameters.

In various embodiments, the present system may be combined into a programming device that applies one or more other methods for defining patterns of neurostimulation pulses. Method 1060 may be performed using a programming device that is also configured to perform one or more other graphical and/or non-graphical methods for defining patterns of neurostimulation pulses. An example of another graphical method for programming the pattern of neurostimulation pulses is discussed in U.S. Provisional Patent Application Ser. No. 62/050,505, entitled “GRAPHICAL USER INTERFACE FOR PROGRAMMING NEUROSTIMULATION PULSE PATTERNS,” filed on Sep. 15, 2014, assigned to Boston Scientific Neuromodulation Corporation, which is incorporated by reference in its entirety. In various embodiments, the user may define the pattern of neurostimulation pulses by using building blocks, such as waveforms and electrode assignments, created by using various methods.

FIG. 11 illustrates an embodiment of a viewing/editing module 1146 providing for various viewing/editing controls using GUI 710. Viewing/editing module 1146 represents an embodiment of viewing/editing module 746. In the illustrated embodiment, viewing/editing module 1146 includes a viewframe zoom in/out control 1170, a single-axis compress/expand control 1171, a multi-axis compress/expand control 1172, an import/export control 1173, a value editing control 1174, a copy/paste control 1175, measurement control 1176, and a script control 1177. FIG. 12 illustrates an embodiment of portions of the screen of GUI 710 displaying buttons allowing for user access to these viewing/editing controls. The controls are illustrated in FIGS. 11 and 12 by way of example, and not by way of limitation. In various embodiments, viewing/editing module 1146 may include any one or any combination of viewframe zoom in/out control 1170, single-axis compress/expand control 1171, multi-axis compress/expand control 1172, import/export control 1173, value editing control 1174, copy/paste control 1175, measurement control 1176, script control 1177, and any other controls that facilitate the definition of the set of one or more stimulation waveforms.

In FIG. 12, a viewing/editing bar 1246 includes viewframe zoom in/out control buttons 1270, single-axis compress/expand control buttons 1271, multi-axis compress/expand control buttons 1272, import/export control buttons 1273, value editing control buttons 1274, copy/paste control buttons 1275, measurement control buttons 1276, and script control buttons 1277, which provide for user access to viewframe zoom in/out control 1170, single-axis compress/expand control 1171, multi-axis compress/expand control 1172, import/export control 1173, value editing control 1174, copy/paste control 1175, measurement control 1176, and script control 1177 of viewing/editing module 1146, respectively. The arrangement for viewing/editing bar 1246 and the control buttons as well as the fields and parameter graphs are illustrated in FIG. 12 by way of example, and not by way of limitation. Any other arrangements on the screen of GUI 710 may be employed without departing from the scope of the present subject matter.

Viewframe zoom in/out control 1170 allows the user to change the scale for the time and the value of the parameter on a displayed parameter graph for viewing (without changing the underlying parameter). The user may use viewframe zoom in/out control buttons 1270 to zoom in for viewing details in a selected small portion of a parameter graph or zoom out for an overview of a large portion of the parameter graph.

Single-axis compress/expand control 1171 allows the user to adjust the parameter graph by compressing or expanding the parameter graph in one dimension (e.g., either the time or the value of the parameter). The compression or expansion changes the parameter represented by the parameter graph (rather than merely changing the scale for viewing). The user may use single-axis compress/expand control buttons 1271 to compress or expand the parameter graph in one dimension. In various embodiments, single-axis compress/expand control 1171 is enabled during both the composition mode and the real-time programming mode in a programing device such as external programmer 602.

Multi-axis compress/expand control 1172 allows the user to adjust the parameter graph by compressing or expanding the parameter graph in multiple dimensions (e.g., both the time and the value of the parameter). The compression or expansion changes the parameter represented by the parameter graph (rather than merely changing the scale for viewing). The user may use multi-axis compress/expand control buttons 1272 to compress or expand the parameter graph in both dimensions. In various embodiments, multi-axis compress/expand control 1172 is enabled during both the composition mode and the real-time programming mode in a programing device such as external programmer 602.

Import/export control 1173 allows the user to import one or more predefined stimulation waveforms and/or parameter graphs into external storage device 618 and export one or more predefined stimulation waveforms and/or parameter graphs from the storage device external storage device 618. The user may use import/export control buttons 1273 to initiate a process of importing the one or more predefined stimulation waveforms and/or parameter graphs from another user or exporting the one or more predefined stimulation waveforms and/or parameter graphs to another user. Such imports and exports enable the user to share stimulation waveforms and/or their building blocks with other users in a community of users that use the same or similar neurostimulation systems to treat patients.

Value editing control 1174 allows the user to enter one or more values for a parameter, when entering values are quicker and easier than, for example, using incremental controls. The user may use value editing control buttons 1274 to start and end adjusting one or more parameters by entering one or more values. In one embodiment, value editing control 1174 is enabled during the composition mode, and disabled during the real-time programming mode, in a programing device such as external programmer 602.

Copy/paste control 1175 allows the user to select and copy all or a portion of a parameter graph and paste it to another temporal location in the parameter graph, or paste it to a parameter graph associated with another field of the one or more stimulation fields. The user may use copy/paste control buttons 1275 to copy the selected portion of a source parameter graph and paste it to a destination parameter graph at a user-selected temporal location.

Measurement control 1176 allows the user to make various measurements on the displayed parameter graphs, in a manner similar to an oscilloscope. The user may use measurement control buttons 1276 to start and end one or more measurements. For example, one or more measurement tools such as a pointer and a caliper may be displayed in response to the “MEASURE ON” button being clicked on. The user may use the pointer to measure the value of the parameter and the time for a location on a parameter graph (such as a parameter curve), and the caliper to measure distance between two locations on a parameter graph or two parameter graphs (e.g., range of values of a parameter and time interval between two points on one or two parameter graphs, such as the time interval between two pulses in two different fields). In one embodiment, measurement control 1176 also derives one or more parameters from the measurements. Examples of measured and derives parameters include averages and standard deviations. In one embodiment, embodiment, measurement control 1176 displays results of measurements on the screen, such as results of measurements selected by the user. In one embodiment, measurement control 1176 displays on the screen a cross-hair that tracks a selected parameter curve.

Script control 1177 performs scripting language conversion to allow the user to define portions of the set of one or more stimulation waveforms using a script. In various embodiments, script control 1177 can translate a script into portions of the set of one or more stimulation waveforms (e.g., one or more parameter graphs). In various embodiments, script control 1177 can also translate portions of the set of one or more stimulation waveforms into a script. The user may use script control buttons 1177 to input a script and converts it into one or more parameter graphs or output a script converted from one or more parameter graphs. In various embodiments, scripting language allows the user to input a prewritten script to specify all variables associated with the set of one or more stimulation waveforms. In various embodiments, GUI 710 can output one or more stimulation waveforms as a script file for modification or transfer to another programming system.

It is to be understood that the above detailed description is intended to be illustrative, and not restrictive. Other embodiments will be apparent to those of skill in the art upon reading and understanding the above description. The scope of the invention should, therefore, be determined with reference to the appended claims, along with the full scope of equivalents to which such claims are entitled.

What is claimed is:

1. A system for delivering neurostimulation using a plurality of electrodes, comprising:
a storage device storing a set of one or more stimulation waveforms each associated with a field of one or more stimulation fields, the one or more stimulation waveforms each representing a pattern of neurostimulation pulses to be delivered during a stimulation period, the one or more stimulation fields each specified by a set of electrodes selected from the plurality of electrodes;
a programming control circuit configured to generate a plurality of stimulation parameters controlling delivery of the neurostimulation pulses according to the set of one or more stimulation waveforms; and
a graphical user interface (GUI) including a composition control circuit configured to define each stimulation waveform of the set of one or more stimulation waveforms as a function of one or more parameter graphs each depicted as a function of time for the stimulation period, the composition control circuit including a waveform definition module configured to:
allow activation of one or more fields of the one or more stimulation fields;
present a parameter graph of the one or more parameter graphs for each field of the activated one or more fields; and
allow for adjustment of the presented each parameter graph.

2. The system of claim 1, wherein the composition control circuit further comprises a field definition module configured to define each field of the one or more stimulation fields by the set of electrodes selected from the plurality of electrodes and a current distribution over the selected set of electrodes.

3. The system of claim 1, wherein the waveform definition module is configured to depict the presented each parameter graph as a curve.

4. The system of claim 3, wherein the waveform definition module is further configured to depict the presented each parameter graph as a pattern of symbols each representing one or more pulses of the neurostimulation pulses.

5. The system of claim 1, wherein the waveform definition module is configured to present the one or more stimulation fields and receive a selection of the one or more fields for activation.

6. The system of claim 5, wherein the waveform definition module is configured allow the presented each parameter graph to be graphically edited.

7. The system of claim 6, wherein the waveform definition module is configured to present a frequency graph representing a pulse frequency.

8. The system of claim 7, wherein the waveform definition module is further configured to present a duration graph representing a pulse width.

9. The system of claim 8, wherein the waveform definition module is further configured to present an amplitude graph representing a pulse amplitude.

10. The system of claim 6, wherein the storage device is configured to store a pattern library including one or more template waveforms, and the waveform definition module is further configured to select a template waveform from the template library as a stimulation waveform of the set of one or more stimulation waveforms or a parameter graph of the one or more parameter graphs.

11. The system of claim 1, wherein composition control circuit is configured to allow for export of a selected portion of the set of one or more stimulation waveforms from the system and import of a waveform into the system to be inserted to the set of the one or more stimulation waveforms.

12. The system of claim 1, further comprising a stimulation device including:
a stimulation output circuit configured to deliver the neurostimulation pulses; and
a stimulation control circuit configured to control the delivery of the neurostimulation pulses using the plurality of stimulation parameters; and
a programming device communicatively coupled to the stimulation device and including the storage device, the programming circuit, and the GUI.

13. The system of claim 12, wherein the stimulation device comprises an implantable stimulator including an implant telemetry circuit configured to receive the plurality of stimulation parameters, and the programming device comprises an external programmer including an external telemetry circuit configured to transmit the plurality of stimulation parameters to the implantable stimulator via a wireless telemetry link.

14. A method for controlling neurostimulation by a user for delivery through a plurality of electrodes, the method comprising:
defining each stimulation waveform of a set of one or more stimulation waveforms as a function of one or more parameter graphs each depicted as a function of time for a stimulation period, the one or more stimulation waveforms each associated with a field of one or more stimulation fields and representing a pattern of neurostimulation pulses to be delivered to the field during the stimulation period, the one or more stimulation fields each specified by a set of electrodes selected from the plurality of electrodes;
allowing the user to activate one or more fields of the one or more stimulation fields using a graphical user interface (GUI);
presenting a parameter graph of the one or more parameter graphs for each field of the activated one or more fields using the GUI;
allowing the user to adjust the presented each parameter graph;
updating the set of one or more stimulation waveforms in response to the adjustment of the presented each parameter graph; and
generating a plurality of stimulation parameters based on the set of the one or more stimulation waveforms, the plurality of stimulation parameters allowing for delivery of the neurostimulation from a stimulation device to be controlled according to the set of the one or more stimulation waveforms.

15. The method of claim 14, wherein defining the each stimulation waveform of the set of one or more stimulation waveforms comprises:
creating one or more template waveforms for the each stimulation waveform; and
selecting a template waveform from the stored one or more waveforms to be used as the each stimulation waveform.

16. The method of claim 15, wherein creating the one or more template waveforms comprises creating one or more therapy-specific template waveforms for one or more types of neurostimulation therapy.

17. The method of claim 14, wherein presenting the each parameter graph comprises presenting one or more of a frequency graph representing a pulse frequency, a duration graph representing a pulse width, and an amplitude graph representing a pulse amplitude.

18. The method of claim 17, wherein presenting the each parameter graph comprises presenting one or more curves.

19. The method of claim 18, wherein presenting the each parameter graph comprises presenting a pattern of symbols each representing one or more pulses of the neurostimulation pulses.

20. The method of claim 14, further comprising:
transmitting the plurality of stimulation parameters to an implantable stimulator; and
controlling delivery of the neurostimulation pulses from the implantable stimulator using the plurality of stimulation parameters.
